(19) United States
(12) Patent Application Publication
(10) Pub. No.: US 2004/0155457 A1
(43) Pub. Date: Aug. 12, 2004

(54) Connecting element comprising a first body and a method for injection moulding a connecting element

(75) Inventors: Signe Thorning Mejlhede, Svinninge (DK); Grete Kornerup, Haslev (DK)

Correspondence Address: MERCHANT & GOULD PC, P.O. BOX 2903, MINNEAPOLIS, MN, 55402-0903, US

(73) Assignee: Maersk Medical A/S, Roskilde (DK)

(21) Appl. No.: 10/437,488

(22) Filed: May 13, 2003

(30) Foreign Application Priority Data
Feb. 12, 2003 (DK) PA 2003 00207

(51) Int. Cl. F16L 55/00

(52) U.S. Cl. 285/21.1; 285/423; 285/285.1

(57) ABSTRACT

The invention relates to a connecting element (1) comprising a first body with a through-going axial opening (3) situated in the first body (2) for liquid communication between the first end portion (4) of the first body (2) and the second end portion (5) of the first body (2), said first end portion (4) comprising a cavity (6), and wherein the cavity (6) comprises a female part (9) with a through-going opening (10) and manufactured from a first plastics material and comprising an inner area (35) for mounting of a tube (24) by means of first attachment means (8); and wherein the first body is manufactured from a second plastics material different firm the first plastics material. Hereby a connecting element is obtained that is not associated with the prior art connecting elements; and wherein it is possible to wipe the connecting element as such with eg ethyl alcohol prior to use, and to use a plastics material, in which stresses are not released during this wiping, while simultaneously it is possible to use a soft glue between the connecting element and the plastics tube, such that leaks do not occur in this are during the physical influencing of the element.

The invention relates to a connecting element comprising a first body with a through-going axial opening situated in the first body for liquid communication between the first end portion of the first body and the second end portion of the first body, said first end portion comprising a cavity. The invention also relates to a method of injection moulding a connecting element in an injection moulding mould, said connecting element comprising a first body with a through-going axial opening situated in the first body for liquid communication between the first end portion of the first body and the second end portion of the first body.

U.S. Pat. No. 5,167,647 teaches a connecting element comprising a tube and wherein a method is taught for moulding the connecting element along with the plastics tube, whereby it is avoided to subsequently have to join the connecting element and plastics tube. Such method can be selected, but seems to be comparatively expensive in connection with the manufacture of the component.

Moreover, it is known to manufacture connecting elements in a single plastics material, and wherein the connecting element comprises an end portion for mounting of a plastics tube by means of a glue within the through-going bore as such, and wherein the second end of the connecting element comprises a luer-lock for connection to for instance an insulin pump. The drawback of such system can be that, in case a wiping is performed with eg ethyl alcohol or the like chemical—which is necessary prior to use of the connecting element, stresses may be released in the plastics material during physical strain thereof, thereby bringing about leaks. By selection of another material which will not release stresses, however, the problem will be encountered that the mounting of the tube to the connecting element by means of a gluing has to be performed with a hard glue with the ensuing risk of the leak occurring therein due to the inflexible connection. It is therefore desirable that the connection between the connecting element and the tube is provided by means of a soft glue, ie a glue that yields to physical impact.

It thus the object of the present invention to provide a connecting element that does not have the drawbacks of the prior art connecting elements, and wherein it is possible to perform a wiping with ethyl alcohol or the like of the connecting element as such prior to use and to use a plastics material in which stresses are not released by this wiping, while simultaneously it is possible to use a soft glue between the connecting element and the plastics tube, such that leaks do not occur in this area during the physical impact on the element.

This object is achieved with a connecting element of the kind described in the introductory part and wherein the cavity also comprises a female part with a through-going opening and manufactured from a first plastics material and comprising an inner area for mounting of a tube, and wherein the first body is manufactured from a second plastics material different from the first plastics material.

The object is also obtained with a method as mentioned above, and wherein a first plastics material is also injected into the mould for providing a female part with a through-going opening; and a second plastics material is subsequently injected into the mould for providing the first body, said first body enclosing at least a portion of the outer faces of the female part; and said first body comprising a through-going opening, whose central axis is axially coincident with the central axis of the female part.

The outer physical shape of the connecting element is thus configured essentially in accordance with known principles. However, the novel aspect is that a moulding thereof is performed in two materials, wherein the outer material as such encloses the side faces of the inner material, such that, during wiping with ethyl alcohol or the like, no connection is provided to the material situated interiorly of the connecting element as such.

Thus, the connecting element consists of an internal female part manufactured from a plastics material that enables the use of an attachment means in the form of a soft glue for mounting of a plastics tube, and wherein this female part which thus has a through-going opening is enclosed completely or partially by a first body as regards its outwardly radially oriented faces. The first body can be made of a plastics material which is resistant to chemicals and able to resist eg wiping with ethyl alcohol. Here, it is important that the cavity in which the tube is mounted is enclosed by the side walls of the female part, and that the glue has, as a minimum, connection between the female part of the plastics tube as such. Should, in the cavity, further glue be applied onto the parts that are thus constituted by the plastics material of the first body, this is of no consequence to the longevity; the important aspect being the connection between the female part and the tube and that this connection has an area—an inner area—wherein an outer wiping of the connecting element with a liquid is unable to reach said area.

It is noted in this context that the through-going opening of the first body has a central axis which is axially parallel and coincident with the central axis of the female part, thereby ensuring liquid transport.

By provision of a connecting element according to the invention and as further featured in claim 2, selection of a convenient material is accomplished, since it is a characteristic feature of thermoplastics that it exhibits three different, more or less sharply delimiting state areas as a function of the temperature, viz a solid state, a rubber elastic state and a viscous state. Upon heating the material becomes soft and assumes a rather more solid structure upon cooling and, in principle, it is possible to shape it several times.

By provision of a connecting element according to the invention and as further featured in claim 3, a molecular structure is accomplished in which the chains are positioned randomly.

By provision of a connecting element according to the invention and as further featured in claim 4, a conveniently amorphous plastics material is selected.

Provision of the connecting element according to the invention and as further featured in claim 5 brings about the advantages associated with crystalline plastics materials. They are characterised in that the molecules are situated closely adjoining each other whereby the intermolecular forces act more strongly and more energy is to be supplied in order to overcome it. In practice this means that crystalline plastics/partially crystalline plastics generally have a higher temperature of vitrification and a higher melting point than amorphous materials. The crystalline areas in the material have a distributing effect on light, and therefore crystalline and partially crystalline plastics have a milky appearance.

By provision of a connecting element according to the invention and as further featured in claim 6 a convenient choice is made as regards crystalline plastics material.

Provision of a connecting element according to the invention and as further featured in claim 7 ensures that the plastics material of the female part cannot be reached during wiping of the connecting element, eg with ethyl alcohol; and due to retention devices being mounted on the outside, good retention is provided between the female part and the first body ensuring that the female part cannot be loosened.

Provision of a connecting element according to the invention and as further featured in claim 8 brings about a convenient shape and structure of the female part.

Provision of a connecting element according to the invention and as further featured in claim 9 brings about a reasonable joining between the connecting element and a plastics tube. Use of a soft glue as first attachment means allows the tube to retain its flexibility in the in-use situation without a risk of leakage.

Provision of a connecting element according to the invention and as further featured in claim 10 brings about an option for connection to eg an insulin pump. It is mentioned herein that a luer-lock has a conical cone with standardised locking adaptation. There are standards for female luers as well as for male luers. In connection with this connecting element, a male luer is preferred.

The invention also relates to an infusion unit manufactured from the connecting element, and wherein the second end portion of the connecting element is connected to an insulin pump by means of the luer lock, and wherein the first end part is connected to a plastics tube by means of a soft glue between female part and the interior delimiting faces of the female part and the upper faces of the tube.

As mentioned above, the invention relates to a method as featured in claim 12.

Claims 13-15 teach convenient steps in connection with the method as such.

Finally the invention relates to use of the method for the manufacture of the connecting element.

The invention will now be explained in further detail with reference to the drawing, wherein

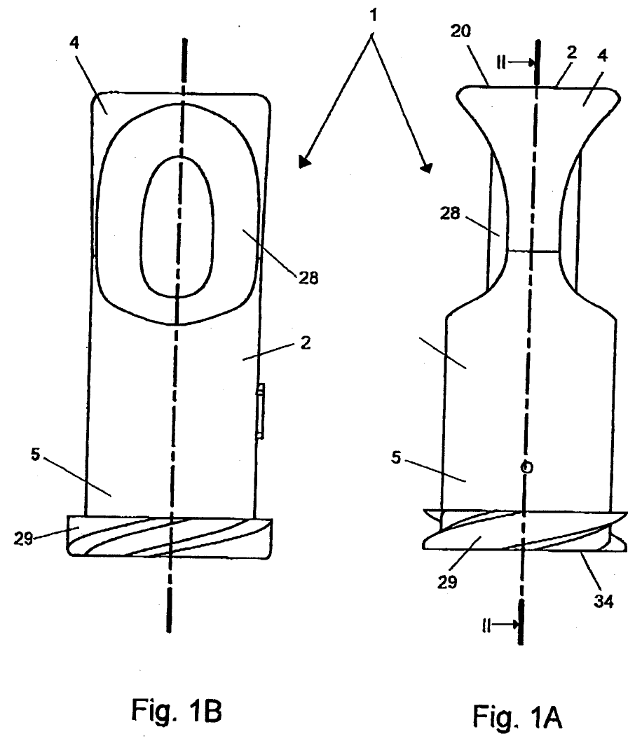

FIGS. 1A-B show an example of a connecting element according to the invention seen from two different sides;

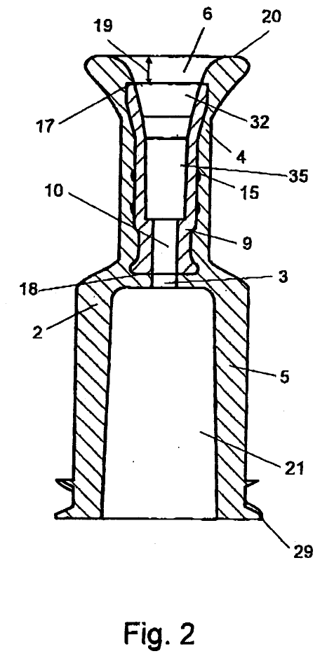

FIG. 2 is a sectional view along II-II in FIG. 1A;

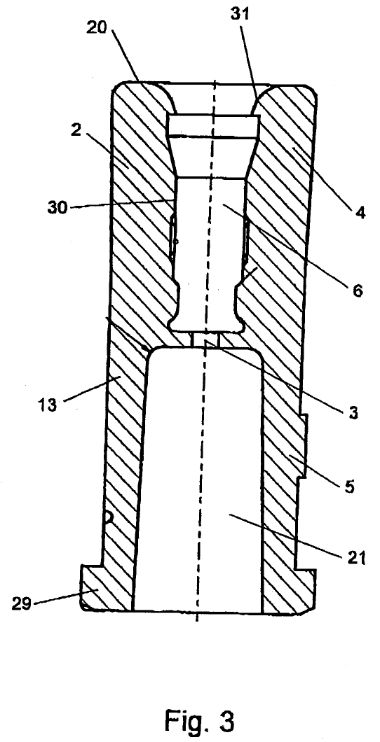

FIG. 3 is a sectional view of the first body in detail form;

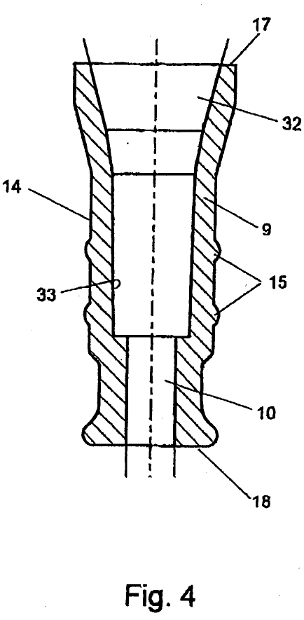

FIG. 4 is a sectional view of the female part in detail form;

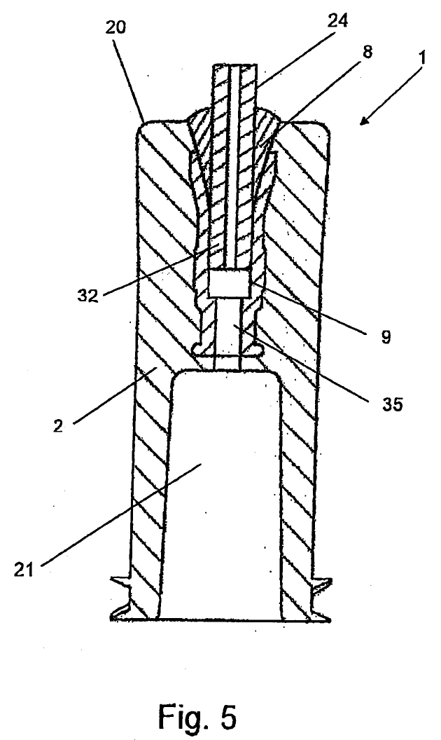

FIG. 5 shows the disclosures of FIG. 2 with plastics tube mounted in the inner area of the female part,

FIGS. 1A and 1B show a connecting element 1, seen from the side in a first position and then turned by 90°. The connecting element—see also FIG. 2—comprises an outer core, also designated the first body which is preferably made of a thermoplastics material that is chemically resistant preferably to ethyl alcohol. This material is referred to as the second plastics material and partially crystalline plastics material is preferred, eg polypropylene. Polypropylene is a partially crystalline plastics material in which the molecule chains are located in parallel with each other and form rather closely packed areas, the so-called crystallites. Polypropylene is a thermoplastics material with an attractive balance between thermal and chemical resistance and good mechanical and electrical properties. Furthermore, polypropylene is immune to bacteria and fungi. Additionally polypropylene is a plastics material having a very wide-ranging resistance to chemicals. It is resistant to salts, acids, bases (inorganic) and alcohols and some oils. In principle, it is the crystallite that is decisive to the resistance to chemicals Also other materials can be used, eg polyamide, polyethylene, poly-ethylene-ether-ketone (PEEK).

The first body 2 comprises a first end portion 4 and a second end portion 5, wherein the first end portion 4 will have a cavity 6 as it is also seen in the sectional view shown in FIG. 2.

The second end portion 5 comprises a luer lock 21, in which a male luer lock is preferably used. Finally, the second end portion 5 comprises an external double-thread 29 for being mounted to preferably an insulin pump. The outer shape of the connecting element 1 is such that it is an essentially cylindrical shape, but wherein the first end portion 4 has a narrowed portion for providing an oval narrowing area 28. This serves to improve the gripping around the connecting element 1 during handling thereof. The first body 2 has an outer, essentially plane upper delimiting face 20. The distance from this and down to the lower delimiting face 34 is within the range 17 mm.

FIG. 2 is a sectional view wherein the joining between the two elements that constitute the connecting element 1, viz the first body 2—also designated outer core—and the female part 9—also designated inner core, will appear. The first body is manufactured such that there is liquid communication throughout its entire passage and comprises axially a passage throughout its entire interior, wherein, in its second end part 5, it has such shape that it forms a luer lock 21; and wherein, from this luer lock 21, there is an axial opening 3 to a second area designated a cavity 6; the inner face of this cavity 5 being congruent with the outer faces of the female part/inner core 9, inasmuch as the faces that are in mutual contact are concerned. The central axis of the axial opening is axially parallel and coinciding with a through-going opening 10 provided interiorly of the female part 9. Opposite this opening there is, in the second end section of the female part, a so-called connector part 32 for the tube and configured with side faces that diverge outwards towards the upper delimiting face 17 of the female part, said face 17 being of plane configuration.

In the area between the tube connector part 32 and the otherwise cylindrically shaped inner opening 10, the interior area 35 and a further, essentially conical connecting area that may also constitute a part of the tube connector part and serve as glue reservoir are situated. The interior area is situated such that the glue ensuring connection between tube and female part cannot be in liquid communication with the exterior of the connecting element. The female part 9 is located such that its upper delimiting face 17 is located a first distance 19 from the upper delimiting face 20 of first body 2 as such. It should further be mentioned that the female portion 9 has, on its outer circumference, retention devices 15, eg in the form of studs or wings that serve as undercuts, whereby good gripping is ensured during moulding between the female part and the surrounding first body 2.

The outer core will be described with reference to FIG. 3. The outer core 2 comprises, as mentioned, an essentially outer cylindrically shaped sleeve, having essentially three cavities; a cavity in the second end portion 5 comprising a luer lock 21 and preferably a male luer lock from which there is connection, via a central opening 3 to the first end part of the first body 2 having a cavity 6, the interior faces of which are shaped to be essentially congruent with the outer faces of the female part. The outer core is manufactured from a chemically resistant material 13, whereby no stresses are released when it is wiped with eg ethyl alcohol.

The inner core or the female part 9 will be described with reference to FIG. 4. It also comprises a sleeve-like construction, its outer walls 14 being essentially cylindrical with various pouches/retention devices mounted thereon for providing good retention between the female part and the outer core.

Its interior cavity comprises essentially four areas, viz a through-going opening 10 in communication with the inner area 35 being a cylindrical area having a length of essentially about 3 mm and with a somewhat larger diameter; a conical area of about 1 mm forming the connection between the interior area, opening area and the tube connector part 32; and said tube connector part constituting the upper part of the female portion. This tube connector part is configured such that the walls diverge outwards towards the upper delimiting face 17 of the female part, said face 17 being of essentially plane configuration. As mentioned, the outer walls of the female part also comprise retention devices 15.

The diameter of the inner area is adapted to the diameter of the selected tube.

The connection between the tube and the female part, including the tube connector part 32 that may also be an area where the glue bonds and seals the remaining entrance, will be described in further detail with reference to FIG. 5. It shows the joint between a connecting element 1 and a tube 24, wherein a section as been made through the connecting element and the tubular element. A luer lock 21 is shown, and opposite that the tubular element is located in the female part/inner core 9 of the connecting element corresponding to the inner area 35. It is noted in this context that the tube connector part 32 is situated such that its upper delimiting face is located at a distance from the upper delimiting face 20 of the first body. When the tube seizes the tube connector part 32, and wherein—between the outer face of the tube 24 and the inner face of the tube connector part 32—attachment means 8 are located in the form of a soft glue, the risk of chemicals, if any, used for wiping the outer face of the first body penetrating downwards to the inner area 35 of the female part as such will be small due, on the one hand, to said distance and, on the other, to the sealing properties of the glue. This is essential since the female part is manufactured precisely from a plastics material that provides good connection to the tube when a soft glue is selected.

This plastics material is not resistant to the material used for wiping. Conversely, for the first body a material can be used that tolerates wiping with chemicals, including eg ethyl alcohol.

It should be noted with regard to soft glue that it is inherently more flexible than hard glue. A soft glue is used precisely in order to enable the plastics tube to retain its flexibility in the in-use situation. After mounting of the glue between the connecting element 1 and the tube 24, a final hardening takes place.

The following physical, mechanical properties apply to a soft glue versus a hard glue.

| | Soft glue | Hard glue |
|---|---|---|
| Extension at break | 120%-240% | 0%-50% |
| Hardness shore D | D25-D60 | D65-D90 |

It should further be mentioned that the tube connector part is to trap the tube and secure the connecting element on the tube until the glue has set. The manufacture as such of the connecting element takes place in that, in a mould, a first plastics material—which is thermoplastics—is injected under a suitable pressure and wherein it is an amorphous plastics material, eg ABS. ABS is made of the monomer components acrylic nitrite, butadiene and styrene. Each of the three structural units adds valuable properties to the material. Acrylic nitrile is responsible for the resistance and dimensional stability in heat. Butadiene equips the material with tenacity and makes it resistant to impacts, while styrene serves to ensure the rigidity of the material and easy processability. ABS tolerates a number of acids, bases and oils with the proviso, however, that the level of internal stresses is very low. There are a number of solvents that can dissolve ABS. Other suitable amorphous materials include eg polycarbonate, polystyrene, acrylic plastics, poly-methyl-methacrylate. This part thus constitutes the female part.

Subsequently, under a second pressure, a suitable pressure, a second plastics material is injected for providing the first body 2, said second plastics material also being a thermoplastics and at least one partially crystalline plastics material, preferably a polypropylene is used. Following such moulding of the connecting element, said moulding taking place eg in accordance with principles like the ones given in patent No. WO 00/73040 and WO 98/35808, an opening of the mould takes place, and the connecting elements are ejected from their cores. Subsequently the assembly process between the connecting element and tube as described above, by means of a soft glue.

It is thus important to the invention that two different plastics materials are used, viz a plastics material for the female part as such that enables gluing between plastics tube and same by means of soft glue, whereby the flexibility of the tube is maintained. It is likewise essential that the material that will be surrounding this connection, viz the first body 2, is manufactured from a material that is chemically resistant, the connecting element being in use able to tolerate the wiping that is performed With chemicals, including eg ether alcohol, without an ensuing release of stresses.

The connecting element with the tube is preferably intended for use for infusion kits, the luer lock being mounted to an insulin pump, while the tube as such is connected to a syringe and an infusion part. It is noted in this context that the soft glue and polypropylene are unable to cooperate. Furthermore it is essential that the plastics chosen for the female part is to be unavailable to the surroundings at least in an area where connection between tube and connecting element is established, such that it is unable to come into contact with chemicals. The type of plastics is selected exclusively for its gluing properties; the glue having, as mentioned, to be a soft glue since a hard glue will destroy the flexibility of the tube.

However, the female part may also have a course, where its upper delimiting edge is flush with the delimiting edge of the first body or even broaches there around and encloses portions of the outer faces of the first body. What matters is that a sealing is provided to prevent the liquid from penetrating from the outside and into the connecting area between tube and the interior area of the female part.

1. A connecting element (1) comprising a first body with a through-going opening (3) situated axially in the first body (2) for liquid communication between the first end portion (4) of the first body (2) and the second end portion (5) of the first body (2), said first end portion (4) comprising a cavity (6), characterised in that the cavity (6) comprises a female part (9) with a through-going opening (10) and manufactured from a first plastics material and comprising an inner area (35) for mounting of a tube (24); and that the first body is manufactured from a second plastics material different from the first plastics material.

2. A connecting element (1) according to claim 1, characterised in that the first as well as the second plastics material is a thermoplastics.

3. A connecting element (1) according to claim 2, characterised in that the first plastics material is an amorphous plastics material.

4. A connecting element (1) according to claim 3, characterised in that the first plastics material is an ABS material.

5. A connecting element (1) according to claim 2, characterised in that the second plastics material is an at least partially crystalline plastics material.

6. A connecting element (1) according to claim 5, characterised in that the second plastics material is a polypropylene.

7. A connecting element (1) according to claim 1, characterised in that the outer walls (14) of the female part (9) are enclosed by the first body (2) and comprises retention devices (15).

8. A connecting element (1) according to claim 7, characterised in that the female part (9) is an essentially rotationally symmetrical body with a rotationally symmetrical through-going opening (3) and an upper delimiting face (17) and a lower delimiting face (18), which upper delimiting face (17) is situated at a first distance (19) from the delimiting face (20) of the first end part (4).

9. A connecting element (1) according to claim 1, characterised in that the tube is mounted by means of first attachment means (8) in the interior area and preferably with a glue.

10. A connecting element (1), characterised in that the second end portion (5) comprises a luer lock (21).

11. An infusion device comprising a connecting element (1) according to claim 1, the second end part (5) of the connecting element (1) being connected to an insulin pump, and said first end portion (4) being connected to a plastics tube (7).

12. A method of injection moulding a connecting element (1) in an injection moulding mould, said connecting element (1) comprising a first body (2) with a through-going axial opening (3) situated in the first body for liquid communication between the first end portion (4) of the first body (2) and the second end portion (5) of the first body (2), characterised in that a first plastics material is injected into the mould for providing a female part (9) with a through-going opening (10), and a second plastics material is subsequently injected into the mould for providing the first body (2), said first body (2) enclosing at least a portion of the outer faces (14) of the female part; and said first body (2) comprising a through-going opening (3), whose central axis is axially coinciding with the central axis of the female part.

13. A method according to claim 12, characterised in that both plastics materials are thermoplastics materials.

14. A method according to claim 12, characterised in that the injected first plastics material comprises an amorphous plastics material, eg an ABS material.

15. A method according to claim 12, characterised in that the second plastics material is an at least partially crystalline plastics material, eg a polypropylene.
